(19) United States
(12) Patent Application Publication
(10) Pub. No.: US 2022/0054317 A1
(43) Pub. Date: Feb. 24, 2022

(54) REUSABLE PERSONAL HYGIENE SWAB FOR PERSONAL HYGIENE OR COSMETICS USE

(71) Applicant: LastSwab ApS, København K (DK)

(72) Inventors: Kåre FRANDSEN, København K (DK); Nicolas AAGAARD, Holte (DK)

(21) Appl. No.: 17/309,984

(22) PCT Filed: Jan. 14, 2020
(86) PCT No.: PCT/EP2020/050768

§ 371 Date: Jul. 7, 2021

(30) Foreign Application Priority Data
Jan. 15, 2019 (DK) PA201900042
Mar. 26, 2019 (DK) PA201900360

(51) Int. Cl. A61F 11/00; A61F 13/38

(52) U.S. Cl. CPC A61F 11/006; A61F 13/38

(57) ABSTRACT

Herein is detailed a reusable personal hygiene swab (1) comprising an elastomeric swab head (10) arranged on a tip (21) of an elongated resilient swab stick (20), wherein the elastomeric swab head (10) is made from an elastomer comprising an abrasive outer surface (12). Thereby reusability and efficiency during use is maintained compared to prior art one-use swabs, such as e.g. cotton swabs.

### TECHNICAL FIELD

The invention disclosed herein generally relates to a personal hygiene swab. More particularly, the present invention relates to a reusable personal hygiene swab suitable for personal hygiene or cosmetics use such as use for cleaning ears, or for cosmetic applications, such as e.g. application of makeup.

### BACKGROUND

Ear swabs, such as cotton swabs (US) or buds (UK), for personal hygiene or cosmetics use are well known examples of one-time use personal hygiene swabs. Such one-time use personal hygiene swabs often have an absorbent cotton swab head covering, usually, both tips of an elongated swab stick, but sometimes covering only one. The latter single-swab head variety is typically used for medical or clinical purposes, where sterility concerns make a single swab head preferable.

For forming a one-time use personal hygiene swab of the prior art, generally an absorbent material, typically cotton, is wrapped over at least one of the tips of an elongated swab stick, thereby forming a one-time use personal hygiene swab comprising at least one swab head on a tip of the elongated swab stick, particularly forming at least one absorbent swab head on a tip of the elongated swab stick.

Conventional cotton swabs are typically made by applying an absorbent cotton covering directly to the tips of the elongated stick, though in some cases, an adhesive is used to hold more firmly the absorbent cotton covering in place at the tip of the elongated swab stick. Unfortunately, the cotton swab may not always be securely attached to the swab stick; and the swab may accidentally fall off, e.g. fall off inside an ear during use. This may cause injuries and infections in the ears if not removed immediately.

Cotton is the absorbent material of choice in practice for several reasons, where with cotton is meant unspun cotton or unspun cotton fibers, not the least availability for cheap in large quantities. However, also for the intended purposes is cotton very suitable. The fibrous nature of the cotton swab will absorb moisture, e.g. from the ear canal, and will also help in providing increased friction for the swab head in the ear canal, thereby providing a mild scraping action against the skin in the ear canal for removing cerumen more mildly than when hard(er), but reusable, ear-scoops or earpicks are used.

Generally, the elongated swab stick is made from resilient materials, with resilient to be understood in the dictionary sense as being a characteristic of materials, which are able quickly to return to their usual shape after having been bent, such materials e.g. including wood, metal, rolled paper or plastic.

However, as existing cotton swabs are for discarding after use, this is the cause of increased materials utilization and waste as global population increases, with the global cotton buds market accounting for more than 540 billion units produced in 2017 (https://www.prnewswire.com/news-releaes/cotton-buds-world-industry-report-2013-2026-analysis-by-material-application-and-geography-300675866.html). Also, when disposing of such cotton swabs, many users discard the swabs to the sewer which causes sewer clogging and pollution and residuals of cotton swabs are commonly found in micro plastics in the oceans due to having been disposed via the sewers.

One-time use personal hygiene swabs also find use in cosmetics as applicators or shapers e.g. of foundation or cosmetics applied to the face to enhance the appearance, or to create an even, uniform color to the complexion. For proper application, sufficient care is required during applying and shaping the makeup in order to achieve an overall smooth, consistent and natural look. However, applying, shaping, and removing foundation or other makeup on the face or skin may require multiple applicators. Obtaining the multiple applicators, for example, makeup swabs/buds, sponges and brushes is costly, as typically the user is required to purchase each applicator individually. In this respect, cotton swabs have been found to constitute a cheap alternative to reusable applicators, this being an additional cause for increased use of such swabs and for the associated increase in pollution from used swabs.

Historically, i.e. before the cotton swab became ubiquitously available, most people on Earth used ear-scoops or earpicks for cleaning their ear canal of cerumen. Such ear-scoops are washable and reusable since they must be manufactured in materials, which allow manufacture of a scoop of some hardness for inserting into the ear canal and for scraping off cerumen from the ear canal walls. Due to genetic differences, cerumen in humans belong to two types, dry and wet cerumen. Typically, people of African or Caucasian descent tend to have wet cerumen, whereas people of Asian descent, including Native Americans, tend to have dry cerumen. For that reason, ear-scoops are still much in use in Asia, whereas the cotton swab has almost completely replaced the ear-scoop in e.g. Europe and North America, due also to cotton's ability to absorb moisture from the wet cerumen. However, at the same time giving rise to the abovementioned problems of lack of reusability of the cotton swab.

Accordingly, there exist a general need for developing efficient reusable personal hygiene swabs for personal hygiene and cosmetic purposes.

In FIGS. 1A and 1B there is detailed a personal hygiene swab (1) of the prior art, such as a cotton swab. The personal hygiene swab (1) comprises a swab head (10) arranged at a tip (21) of an elongated resilient swab stick (20). Typically, the personal hygiene swab (1) will be rotationally symmetrical around a central axis (A-A) both for the safety of a person using the personal hygiene swab (1) e.g. for cleaning an ear canal of cerumen, and for reasons of low manufacturing costs. As the elongated resilient swab stick (20) may comprise two respective tips (21a,21b), such as a first tip (21a) and a second tip (21b), the personal hygiene swab (1) may comprise a first and a second swab head (10a, 10b) arranged at a respective first and second tip (21a,21b) of the elongated resilient swab stick (20).

In the art, numerous examples of reusable personal hygiene swabs for use in personal hygiene and cosmetics have been suggested.

In U.S. Pat. No. 3,871,375 to Bennett, a molded, unitary, double-ended personal hygiene swab formed from a resilient polymer foam is suggested. The polymer foam allows for direct molding of the reusable swab as a unitary unit comprising soft, porous, ellipsoidal ends with open cavities exposed on the external surfaces of the ellipsoidal ends, joined by a central swab stick rendered less resilient and denser, for permitting handling of the personal hygiene swab, by the mold preventing the foam from expanding during formation. The advantage of the reusable personal hygiene swabs disclosed in Bennett is ease of manufacture while maintaining beneficial elements of the cotton swab, such as e.g. the ability to absorb moisture and provide a mild scraping of the ear canal, caused by the porous structure of the polymer foam. However, as the pores penetrate into the foam, cleaning becomes difficult with time, limiting reusability.

In US 2003/0181933 A1 to Eicoff there is suggested a reusable ear cleaning apparatus comprising a tip having protrusions for removing earwax by abrasion. The protrusions disclosed in Eicoff may vary in size and shape and a tip having protrusions may comprise both small and large protrusions.

The protrusions may be constructed from a pliable material, such as, for example rubber, plastic, cotton paper or the like. Further, the protrusions may be constructed with the same material used to construct the tip, or from a different material.

Both in Bennett and in Eicoff, solutions are sought to the problem of removing cerumen from the ear canal by abrasion using a personal hygiene swab without the swab head comprising a fibrous material for forming an abrasive surface on the swab head. However, a drawback to reusable swab heads made from e.g. rubber is that cerumen (earwax) will coat the reusable swab head and reduce the friction of the abrasive surface to a level, which can prevent further use of the swab head until cleaned of all cerumen, making repeated washing necessary.

The present inventors, having regard to the prior art, have surprisingly discovered that a number of the shortcomings to reusability of the reusable personal hygiene swabs of the prior art can be overcome by a mild spark erosion treatment of the reusable swab head, thereby creating a reusable swab head comprising an abrasive outer surface having a surface roughness characterized by having a greatest roughness height, R_{z}, below 500 μm and an arithmetical mean roughness, Rₐ, below 100 μm. Thereby the reusable swab head remains slightly rough irrespective of cerumen coating the swab head and hence able to perform its (mild) abrasive action on the inside of the ear canal. Likewise, when used as a cosmetics swab, the slight roughness helps retaining, removing and/or distributing cosmetics and like.

### SUMMARY OF THE INVENTION

In accordance with the invention, there is herein disclosed in a first aspect and embodiment, a reusable personal hygiene swab (1) comprising an elastomeric swab head (10) arranged on a tip (21) of an elongated resilient swab stick (20), wherein the elastomeric swab head (10) is made from an elastomer comprising an abrasive outer surface (12) having a surface roughness characterized by a greatest roughness height, R_{z}, below 500 μm and an arithmetical mean roughness, Rₐ, below 100 μm, as determined in accordance with BS EN ISO 4287:2000.

In a second embodiment of the first embodiment, a reusable personal hygiene swab (1), wherein the greatest roughness height, R_{z}, preferably is below 175 μm, 150 μm, 125 μm, or most preferred below 100 μm, or below 75 μm.

In a third embodiment of the previous embodiments, a reusable personal hygiene swab (1), wherein the arithmetical mean roughness, Rₐ, shall be below 75 μm, below 60 μm, more preferably below 50 μm, below 40 μm, or below 30 μm.

In a fourth embodiment of the previous embodiments, a reusable personal hygiene swab (1), wherein both the greatest roughness height, R_{z}, and the arithmetical mean roughness, Rₐ, are adjusted simultaneously, such that R_{z} is below 200 μm, when Rₐ is below 100 μm, R_{z} is below 150 μm, when Rₐ is below 75 μm, R_{z} is below 100 μm, when Rₐ is below 50 μm, or more preferably, R_{z} is below 75 μm, when Rₐ is below 40 μm.

In a fifth embodiment of the previous embodiments, a reusable personal hygiene swab (1), wherein the elongated resilient swab stick (20) comprising a tip (21) for receiving a swab head (10), further comprises a handle (23) and an extension (22) connecting the tip (21) and the handle (23).

In a sixth embodiment of the fifth embodiment, a reusable personal hygiene swab (1), wherein the handle (23) has a geometry different from the extension (22) and tip (21).

In a seventh embodiment of the previous embodiments, a reusable personal hygiene swab (1), wherein the elastomeric swab head (10) is manufactured from one of rubber, silicone rubber, neoprene rubber, or thermoplastic elastomer (TPE).

In an eighth embodiment of the previous embodiments, a reusable personal hygiene swab (1), wherein the swab head (10) is droplet or tear shaped.

In a ninth embodiment of the previous embodiments, a reusable personal hygiene swab (1), wherein the swab head (10) comprises a blunt swab head tip (11a), preferably a rounded or semi-spherical swab head tip (11a).

In a tenth embodiment of the previous embodiments, a reusable personal hygiene swab (1), wherein the swab head (10) comprises a pointed swab head tip (11b).

In an eleventh embodiment of the previous embodiments, a reusable personal hygiene swab (1), wherein the swab head (10) comprises a plurality of protrusions (13), thereby forming a plurality of grooves (131) and ridges (132).

In a twelfth embodiment of the eleventh embodiment, a reusable personal hygiene swab (1), wherein the plurality of protrusions (13) are bubble-shaped.

In a thirteenth embodiment of the previous embodiments, a reusable personal hygiene swab (1), wherein the plurality of grooves (131) and ridges (132) are a plurality of larger and smaller respectively grooves (131) and ridges (132).

In a fourteenth embodiment of the previous embodiments, a reusable personal hygiene swab (1), wherein the wherein the larger grooves (131) and ridges (132) have a width of between 1 to 1.5 mm, and/or wherein the smaller grooves and ridges have a width of between 0.7 to 1 mm.

In a fifteenth embodiment of the previous embodiments, a reusable personal hygiene swab (1), wherein the swab head (10) comprises a stop (14).

In a sixteenth embodiment of the previous embodiments, a reusable personal hygiene swab (1), wherein the abrasive outer surface (12) is a spark eroded outer surface (12).

In a seventeenth embodiment of the previous embodiments, a reusable personal hygiene swab (1) comprising respectively a first and a second swab head (10a,10b) arranged respectively on a respective first and second tip (21a,21b) of the elongated resilient swab stick (20).

In an eighteenth embodiment of the previous embodiments, a reusable personal hygiene swab (1), wherein the first swab head (10a) comprises a blunt swab head tip (11a), and the second swab head (10b) comprises a pointed swab head tip (11b).

In a nineteenth embodiment of the previous embodiments, a reusable personal hygiene swab (1), wherein the second swab head (10b) is an earpick or ear-scoop.

In a twentieth embodiment of the previous embodiments, a reusable personal hygiene swab (1), wherein the second swab head (10b) is a flare-shaped swab head (10c).

In a twenty-first embodiment of the previous embodiments, a reusable personal hygiene swab (1), wherein the second swab head (10b) comprises a smooth outer surface (12b).

### BRIEF DESCRIPTION OF THE DRAWINGS

FIG. 1: Prior art personal hygiene swab.

FIG. 2: Reusable personal hygiene swab—face-on.

FIG. 3: Reusable personal hygiene swab—cross-section.

FIG. 4: Reusable personal hygiene swab with pointed tip.

FIG. 5: Reusable personal hygiene swab with earpick.

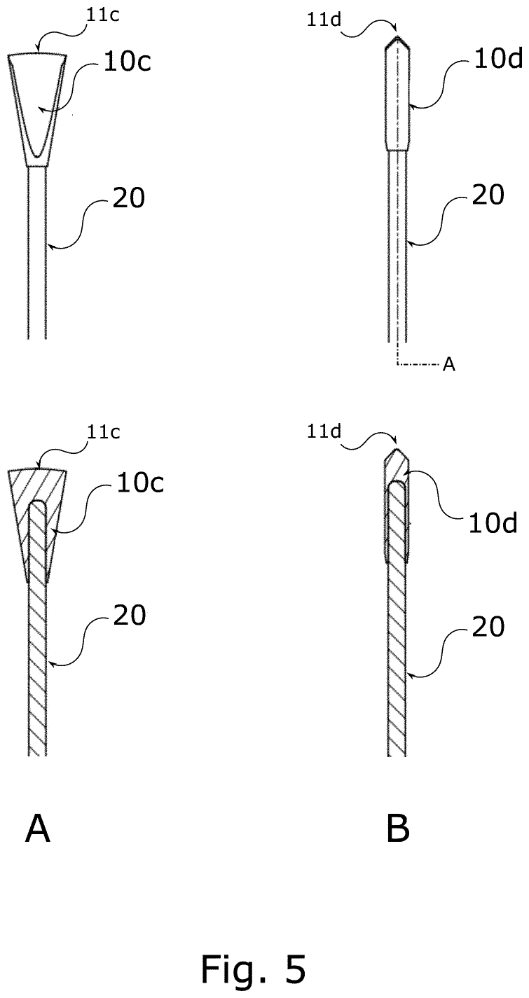

FIG. 6: Reusable personal hygiene swab with stop.

### DETAILED DESCRIPTION

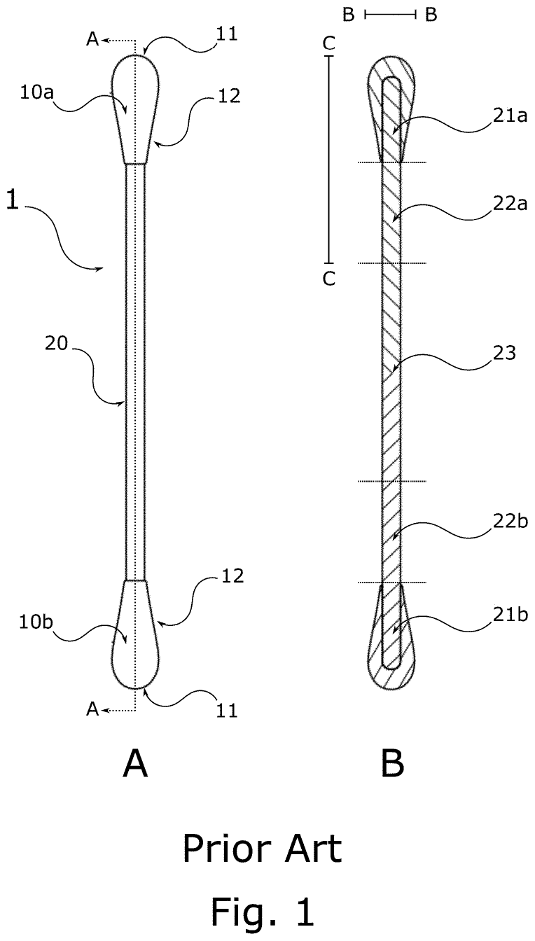

In FIGS. 1A and 1B there is detailed a personal hygiene swab (1) of the prior art, such as a cotton swab. The personal hygiene swab (1) of the prior art can be a reusable personal hygiene swab (1). In FIG. 1A, the personal hygiene swab (1) is shown face-on, in FIG. 1B, the personal hygiene swab (1) is shown in cross-section.

The personal hygiene swab (1) comprises a swab head (10) arranged on a tip (21) of an elongated resilient swab stick (20). As the elongated resilient swab stick (20) may comprise two respective tips (21a,21b), such as a first tip (21a) and a second tip (21b), the personal hygiene swab (1) may comprise a first and a second swab head (10a, 10b) arranged on a respective first and second tip (21a,21b) of the elongated resilient swab stick (20).

The elongated resilient swab stick (20) can tentatively be sectioned into three sections of interest, a tip (21) for receiving a swab head (10), an extension (22) for extending the tip (21) comprising the received swab head (10) into the ear canal, and a handle (23) for allowing a person using the personal hygiene swab (1) to hold and manipulate the personal hygiene swab (1), e.g. while inserted into the ear canal.

Typically, the tip (21), the extension (22) and the handle (23) are comprised into the elongated resilient swab stick (20) as a single geometry, such as e.g. a single elongated cylinder, which is cost efficient; however nothing prevents a reusable personal hygiene swab (1) from e.g. having a personalized handle (23) of a geometry different from the extension (22) and tip (21), since the added cost of personalizing the handle (23) can be offset by the benefit of reusing the personal hygiene swab (1).

For best use, the swab head (10) is arranged coaxially along a central axis (A-A) with at least the tip (21) and the extension (22), but usually also with the handle (23), particularly when the elongated resilient swab stick (20) is of a single geometry. Typically, the personal hygiene swab (1) will be rotationally symmetrical around the central axis (A-A) both for the safety of a person using the personal hygiene swab (1) e.g. for cleaning an ear canal of cerumen, and for reasons of low manufacturing costs.

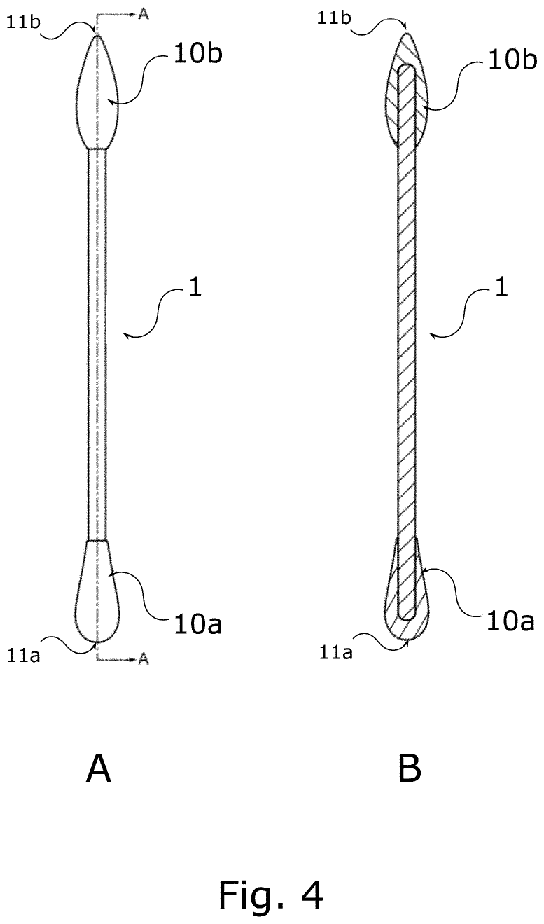

Generally in the prior art, and in some embodiments of the present invention, the swab head (10) is droplet or tear shaped, c.f. e.g. FIG. 1 or FIG. 4, presenting in use an essentially blunt swab head tip (11a), such as e.g. a rounded or semi-spherical swab head tip (11a), inwards in the ear canal towards the tympanic membrane, for avoiding damage if the swab head tip (11a) should contact the tympanic membrane during use of the personal hygiene swab (1). In other embodiments, c.f. e.g. FIG. 4, the swab head tip (11) may be pointed (11b). A pointed swab head tip (11b) is preferable for make-up use, as the pointed swab head tip (11b) e.g. better finishes eyeliner than a blunt swab head tip (11a).

For avoiding damage to the ear canal when inserting the personal hygiene swab (1) into the same, the cross-section (B-B) of the elements (10,21,22) of the personal hygiene swab (1) for insertion into the ear canal must be smaller than the ear canal. Typically, e.g. when the swab head (10) is droplet or tear shaped, the swab head (10) has a cross-section (B-B) at its broadest smaller than 5-6 mm, and the tip (21) and the extension (22) are smaller than the broadest cross-section of the swab head (10), such as e.g. 2-3 mm, depending on the material selected for manufacturing the elongated resilient swab stick (20). Sometimes, often depending on desired manufacturing cost level, the extension (22) may be covered by the swab head (10), effectively having the extension (22) forming part of the tip (21).

Together, a length (C-C) defined by the swab head (10), tip (21), and extension (22), must be sufficient for allowing the personal hygiene swab (1) to be inserted into the ear canal. For adult use of, the length (C-C) of personal hygiene swabs (1), generally permits inserting the personal hygiene swabs (1) into the user's outer ear canal as well as the user's inner ear canal, hence the length (C-C) is such that the swab head tip (11) may actually touch the user's tympanic membrane by accident.

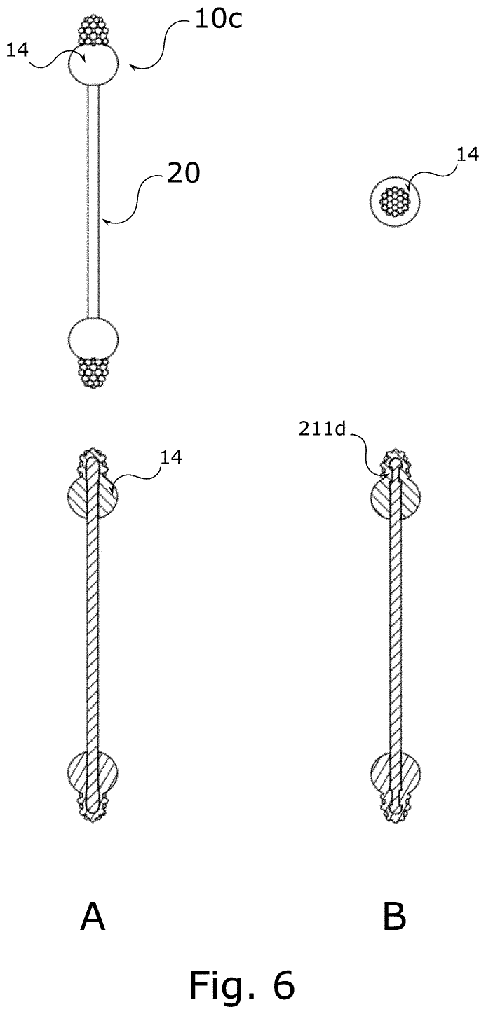

For safety, c.f. e.g. FIG. 6, particularly for children's safety, the swab head (10) often comprises a stop (14), such as e.g. a bulge, having a cross-section larger than the expected cross-section of a user's, particularly a child's, ear canal, limiting the length (C-C) for preventing insertion of the personal hygiene swabs (1) longer into the child's ear than the child's outer ear canal. In these embodiments, the extension (22) is comprised in the tip (21), permitting the swab head (10) comprising a stop (14) to cover the tip (21) comprising the extension (22) completely. Typically, in these embodiments, the limited length (C-C) is 5-6 mm. The full length (C-C) of swab head (10) comprising the stop (14) depends on the chosen stop (14) but is commonly 14-16 mm, when the swab head (10) and stop (14) are cotton.

In a first aspect and embodiment of the present invention, there is detailed a reusable personal hygiene swab (1) comprising an elastomeric swab head (10) arranged on a tip (21) of an elongated resilient swab stick (20), wherein the elastomeric swab head (10) is made from an elastomer comprising an abrasive outer surface (12) having a surface roughness characterized by a greatest roughness height, R_{z}, below 500 μm and an arithmetical mean roughness, Rₐ, below 100 μm, as determined in accordance with BS EN ISO 4287:2000.

In the context of the present invention, a reusable personal hygiene swab (1) shall be understood to be a personal hygiene swab (1), which after a first use, which can encompass multiple individual actions of use forming the first use, can undergo a process of cleaning, such as e.g. washing with water and soap, after which it is returned to a cleaned condition essentially identical to its condition before the first use. Personal hygiene swabs not intended for reuse have a deteriorating condition between a first and a subsequent use, even if cleaned, and are therefore not reusable in the sense of the invention.

In relation to the present invention, an elastomer is a polymer that displays rubber-like elasticity in accordance with IUPAC's definition. Suitable elastomers in relation to the present invention can e.g. be, without preference, rubber, including natural rubber, silicone rubber, latex, neoprene rubber, polybutadiene, butyl rubber, styrene-butadiene rubber, nitrile rubber (Buna N rubber), ethylene-propylene rubber, polyacrylic rubber, thermoplastic elastomers (TPE), fluorosilicone rubber, or e.g. fluoroelastomer rubber, including perfluoroelastomer rubber. The present inventors have obtained suitable results for the elastomeric swab head (10) using natural rubbers, silicone rubbers, neoprene rubbers and different thermoplastic elastomers (TPE), which have all shown to provide suitable abrasive outer surfaces conferring to the desired roughness, when spark eroded as detailed below. Particular preferred are TPE thermoplastic elastomers, such as traditional TPE Classes comprising styrenics (S-TPE's), copolyesters (COPE's), polyurethanes (TPU's), polyamides (PEBA's), and/or polyolefin blends (TPO's) or alloys (TPV's).

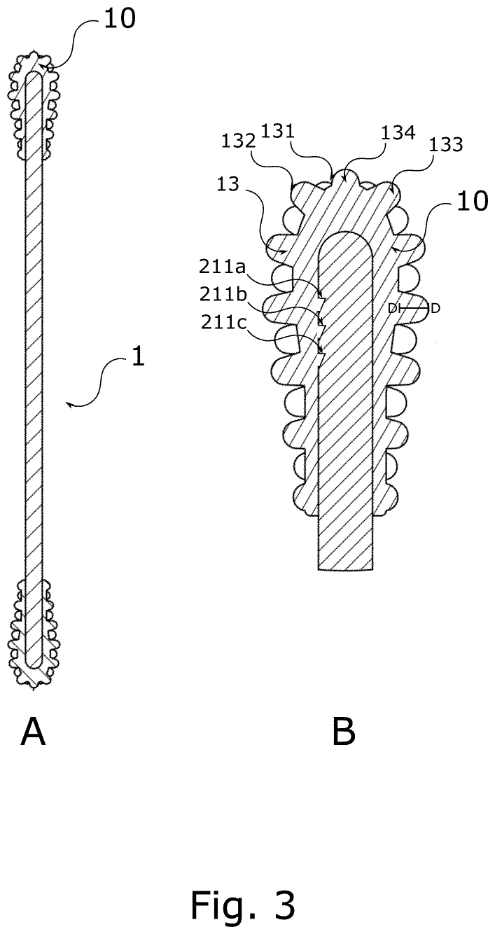

For manufacturing the elongated resilient swab stick (20) for use with the present invention, a plastics material should be used. As not all elastomers suitable for forming a swab head (10) of the invention may be compatible with a specific plastics used in forming the swab stick (20), in some embodiments the tip (21) for receiving a swab head (10) may comprise one or more retaining members (211), such as e.g. one or more barbs (211a-c), c.f. FIG. 3B or an indent (211d), c.f. FIG. 6D. Many times, though, which is a particular advantage of thermoplastic elastomers (TPE)'s, the swab head (10) and the swab stick (20) can be formed in a single manufacturing step, e.g. using injection molding, where local temperature differences in the mold during forming will change the elasticity of the TPE, such that parts of the formed object (e.g. the swab head) remain elastomeric, whereas other parts, (e.g. the swab stick) hardens and loses its elastomeric properties, while instead obtaining an increase in resiliency.

In the context of the present invention, the elastomeric swab head (10) presents an abrasive outer surface (12) for being suitable for performing an abrasive action on e.g. the ear canal walls of the ear for removing cerumen from the ear canal. The outer surface (12) of the swab heads (10) of the invention are essentially continuous outer surfaces, contrary e.g. to the reusable swabs detailed in Bennett, which are foams and therefore do not present an outer surface, but rather a plurality of interlinked abrasive fibers forming an outer, or the fibrous cotton swabs, wherein each fiber has its own outer surface, distinct from all other fiber's surfaces. The advantage thereof is that reusability of the present personal hygiene swabs are increased by presenting an essentially continuous outer surface, as it allows simple cleaning using e.g. water and soap without risk of leaving e.g. cerumen or make-up in pores below the abrasive outer surface after interaction e.g. with the ear canal walls or eyeliner.

In the context of the present invention, the elastomeric swab head (10) presents an abrasive outer surface (12) having a surface roughness characterized by a greatest roughness height, R_{z}, below 500 μm and an arithmetical mean roughness, Rₐ, below 100 μm determined in accordance with BS EN ISO 4287:2000.

To obtain such abrasive outer surfaces (12) having the requisite surface roughness on the elastomeric swab head (10) of the invention, the present inventors have found that spark erosion is very suitable with the tested elastomers (rubber, silicone rubber, neoprene rubber, and thermoplastic elastomer TPE) for obtaining the modified swab heads after initial swab head formation using e.g. injection molding. Accordingly, in one embodiment, the elastomeric swab head (10) comprises a spark eroded outer surface (12).

Spark erosion is a well-known technique for modifying surfaces, which erodes surfaces by ejection crater forming on the surface using electrical sparks. The particulars of spark erosion per se are outside the present invention and are considered well known to the skilled person in the art of spark erosion. In particular, for forming an ejection crater of a given depth and roughness, a given apparatus having a given spark tool requires settings particular to the given apparatus and spark tool for obtaining a given surface of a given roughness. Hence, it is considered in the context of the present invention, that person skilled in operating a given spark erosion apparatus is also skilled in obtaining surfaces of a given roughness using the given apparatus.

Another possible process for post-molding formation of an elastomeric swab head having the required roughness can be laser ablation, where thermal ablation, rather than electrical ablation, causes ejection crater formation and surface roughening. In general, the ejection crater (due to its bowl-like shape) is an easy to clean structure, which forms part of the suitability of the above methods for use with the present invention.

Nevertheless, it is also possible to manufacture surfaces having the required roughness directly in the molding of the elastomeric swab head (10). When injection molding, other surface structures than e.g. the ablation craters form spark erosion, can be used to provide surface roughness to the elastomeric swab heads (10) of the invention, however care must be taken to retain ease of washing.

In accordance with the invention, the abrasive outer surface (12) has a surface roughness characterized by a greatest roughness height (or maximum height of profile), R_{z}, below 500 μm and an arithmetical mean roughness, Rₐ, below 100 μm, as determined in accordance with BS EN ISO 4287:2000. The choice of BS EN ISO 4287:2000 will be familiar to the skilled person as reflecting current industry standards for measuring and reporting surface roughness, Rₐ, and greatest roughness heights, R_{z}.

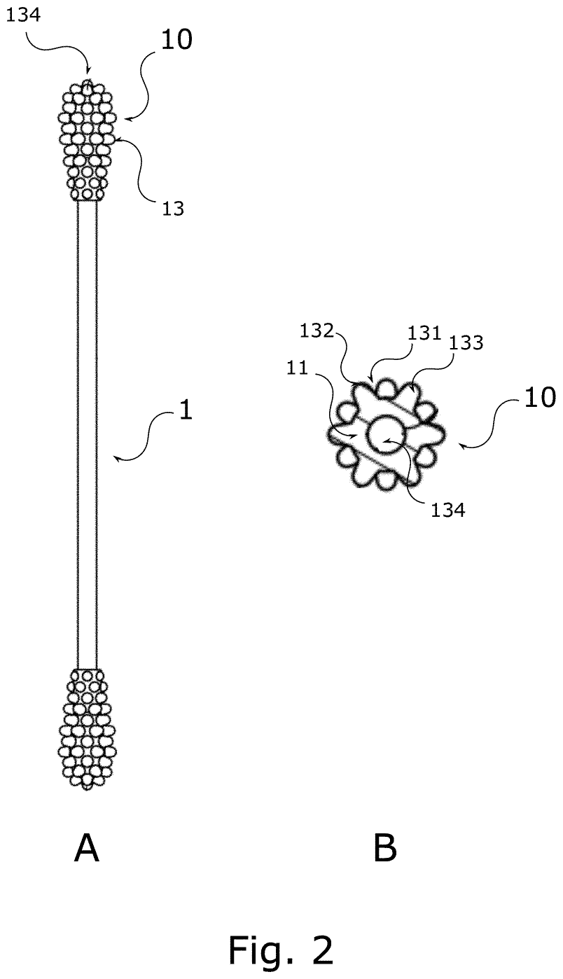

In accordance with the invention, the greatest roughness height (or maximum height of profile), R_{z}, shall be below 500 μm. Usually, it will be smaller, but as shown in FIG. 2, the elastomeric swab head (10) may comprise a plurality of protrusions (13) as will be further detailed below, and one way of manufacturing these protrusions (13) can be by spark erosion, whereby R_{z} is considerably increased. It is however preferred that the greatest roughness height, R_{z}, shall be below 200 μm, and that protrusions (13) comprised on the elastomeric swab head (10) are formed when forming the swab head (10). Thereby sharp edges, which may cut or otherwise damage the skin of the ear canal walls, are avoided.

In embodiments of the invention, the greatest roughness height, R_{z}, preferably is below 175 μm, 150 μm, 125 μm, or most preferred below 100 μm, or below 75 μm.

In accordance with the invention, the arithmetical mean roughness, Rₐ, shall be below 100 μm. Thereby the swab head (10) will be suitable for performing a mild abrasive cleaning of the skin of the ear canal walls without damaging the same. Preferably, the arithmetical mean roughness, Rₐ, shall be below 75 μm, below 60 μm, more preferably below 50 μm, below 40 μm, or below 30 μm.

In accordance with the invention, preferably both the greatest roughness height, R_{z}, and the arithmetical mean roughness, Rₐ, are adjusted simultaneously, such that R_{z} is below 200 μm, when Rₐ is below 100 μm, R_{z} is below 150 μm, when Rₐ is below 75 μm, R_{z} is below 100 μm, when Rₐ is below 50 μm, or more preferably, R_{z} is below 75 μm, when Rₐ is below 40 μm.

Referring to FIGS. 2 and 3, an embodiment of the present invention is shown; wherein the swab head (10) comprises a plurality of protrusions (13), thereby forming a plurality of grooves (131) and ridges (132) for enhancing the abrasive action of the swab head (10) and for smoothly dislodge cerumen or earwax in the ear canal by means of the plurality of grooves (131) and ridges (132) in accordance with the prior art. In FIG. 2A, the reusable personal hygiene swab (1) and swab head (10) is shown face-on, in FIG. 2B, the swab head (10) is shown in along the axis A-A. In FIG. 2A, the reusable personal hygiene swab (1) and swab head (10) is shown in cross-section, whereas in FIG. 3B, an enlarged swab head (10) is shown, also in cross-section. For safety, the plurality of protrusions should comprise a blunt swab protrusion tip (134) arranged at the topmost part of the swab head tip (11). When the swab heads (10) of the invention comprises a stop (14), the stop (14) preferably does not comprise protrusions (13) as the stop (14) is not intended for entry into an ear canal.

In an embodiment, the protrusions (13) are bubble-shaped for avoiding abrasion damage to the skin of an ear canal. In an embodiment, a protrusion has a thickness (D-D) of about 0.5 mm. In an embodiment, a protrusion (13) comprises an essentially bubble-shaped, i.e. a semispherical, termination (133).

In one embodiment, the plurality of grooves (131) and ridges (132) on the swab head (10) comprises an arrangement of larger grooves (131) and ridges (132), and smaller grooves (131) and ridges (132), formed from protrusions (13) of different size. In one embodiment, the arrangement of respectively larger and smaller grooves (131) and ridges (132) are formed alternately with each other. In one embodiment, the larger grooves (131) and ridges (132) have a width of between 1 to 1.5 mm, and/or the small grooves (131) and ridges (132) have a width of between 0.7 to 1 mm. In one embodiment, the larger grooves (131) and ridges (132) have a width of, but not limited to, 1.22 mm. In one embodiment, the smaller grooves (131) and ridges (132) have a width of, but not limited to, 0.81 mm. In one embodiment, when the protrusions (13) are terminated by an essentially bubble-shaped termination (133), the arrangement of grooves (131) and ridges (132) in itself has an appearance resembling an arrangement of bubbles on a surface.

In embodiments of the invention, the second swab head (10b) may be an earpick or ear-scoop arranged on a respective second tip (21b).

In embodiments of the invention for make-up use, the second swab head (10b) may be a flared-shaped swab head (10c) or a sharp, pointed swab head (10d), a respective swab head (10c,10d) comprising respectively a smooth surface or an abrasive surface as detailed herein. In particular, the flared-shaped swab head (10c) may comprise a spark-eroded surface.

### CLOSING COMMENTS

The term “comprising” as used in the claims does not exclude other elements or steps. The term “a” or “an” as used in the claims does not exclude a plurality. A single processor or other unit may fulfill the functions of several means recited in the claims. A reference sign used in a claim shall not be construed as limiting the scope.

Although the present invention has been described in detail for purpose of illustration, it is understood that such detail is solely for that purpose, and variations can be made therein by those skilled in the art without departing from the scope of the invention.

1. A reusable personal hygiene swab comprising an elastomeric swab head arranged on a tip (21) of an elongated resilient swab stick, wherein the elastomeric swab head is made from an elastomer comprising an abrasive outer surface having a surface roughness characterized by a greatest roughness height, R_{z}, below 500 μm and an arithmetical mean roughness, Rₐ, below 100 μm, as determined in accordance with BS EN ISO 4287:2000.

2-4. (canceled)

5. The reusable personal hygiene swab according to claim 1, wherein the elongated resilient swab stick comprising a tip for receiving a swab head, further comprises a handle and an extension connecting the tip and the handle.

6. The reusable personal hygiene swab according to claim 5, wherein the handle has a geometry different from the extension and tip.

7. The reusable personal hygiene swab according to claim 1, wherein the elastomeric swab head is manufactured from one of rubber, silicone rubber, neoprene rubber, or thermoplastic elastomer (TPE).

8. The reusable personal hygiene swab according to claim 1, wherein the swab head is droplet or tear shaped.

9. The reusable personal hygiene swab according to claim 1, wherein the swab head comprises a blunt swab head tip.

10. The reusable personal hygiene swab according to claim 1, wherein the swab head comprises a pointed swab head tip.

11. The reusable personal hygiene swab according to claim 1, wherein the swab head comprises a plurality of protrusions, thereby forming a plurality of grooves and ridges.

12. The reusable personal hygiene swab according to claim 11, wherein the plurality of protrusions are bubble-shaped.

13. The reusable personal hygiene swab according to claim 11, wherein the plurality of grooves and ridges are a plurality of larger and smaller respectively grooves and ridges.

14. The reusable personal hygiene swab according to claim 11, wherein the wherein the larger grooves and ridges have a width of between 1 to 1.5 mm, and/or wherein the smaller grooves and ridges have a width of between 0.7 to 1 mm.

15. The reusable personal hygiene swab according to claim 11, wherein the swab head comprises a stop.

16. The reusable personal hygiene swab according to claim 1, wherein the abrasive outer surface is a spark eroded outer surface.

17. The reusable personal hygiene swab according to claim 1, comprising respectively a first and a second swab head arranged respectively on a respective first and second tip of the elongated resilient swab stick, wherein the first swab head comprises a blunt swab heat tip, and the second swab head comprises a pointed swab head tip.

18-20. (canceled)

21. The reusable personal hygiene swab according to claim 17, wherein the second swab head comprises a smooth outer surface.
